(19) United States
(12) Patent Application Publication
(10) Pub. No.: US 2025/0277207 A1
(43) Pub. Date: Sep. 4, 2025

(54) One or Multiple-Steps Extraction of Nucleic Acids and other Biomolecules using a Cloudy Precipitated Solution

(71) Applicant: Pradheep Chhalliyil, Peoria, AZ (US)

(72) Inventor: Pradheep Chhalliyil, Peoria, AZ (US)

(21) Appl. No.: 18/592,758

(22) Filed: Mar. 1, 2024

(51) Int. Cl. C12N 15/10

(52) U.S. Cl. CPC C12N 15/1013; C12N 15/1017

(57) ABSTRACT

The invention utilizes a single Cloudy-Precipitated Buffer solution (CP Solution) for extracting biomolecules, including nucleic acids. The CP Solution remains cloudy even when heated, becoming clear only with the addition of organic solvents. It can be used in its cloudy state in both One-Step-Method and Multiple-Steps-Method, collectively referred to as OM Method. OM Method involves treating samples with various buffer compositions under optimized conditions, available as a kit. The CP Solution, containing anionic, cationic, and non-ionic detergents, chaotropic agents, alcohols, salts, oils, adsorbents, and organic solvents, does not require heating or organic solvents to clear. It effectively lyses cells and extracts nucleic acids without the need for multiple buffers or additional chemicals. This innovation reduces costs, labor, time, and errors, while minimizing pollution. The invention includes various buffer compositions for nucleic acid extraction and a corresponding kit, addressing current extraction process challenges.

### BACKGROUND OF THE INVENTION

Nucleic Acids (NA) is classified as Deoxyribose Nucleic Acid (DNA) or Ribonucleic Acid (RNA). These nucleic acids are chemical forms of genetic information in most living organisms such as plants, animals, bacteria and fungi while RNA remains as genetic material in some viruses. They determine the hereditary characteristics of every living organism and occur in various forms as condensed chromosomal form or free with or without a cell-membrane boundary or in organelles like mitochondria, chloroplast, or in the cytoplasm as plasmids. They also occur as cell-free DNA or RNA in biological fluids.

These molecules, therefore, serve as tools for diagnostics in clinical and food sectors, agriculture, and the environment, and also in research to understand the fundamentals of life and also in its application in biotechnology industries. Extraction of nucleic acids and subjecting to various Molecular Biology applications or techniques and tools like PCR, isothermal amplification, and sequencing help in the improvement of human life and the planet. Single-sample extraction or automation can be employed to handle a large number of samples.

However, there are many challenges in nucleic acid extractions. These include but are not limited to breaking tough cells in samples especially gram-positive bacteria, fungi, and yeast, removal of contaminating cellular components, removal of cellular micro and macro molecules that otherwise inhibit PCR and other molecular applications, chemicals used in extraction that co-elutes with nucleic acids and inhibit molecular applications, use of expensive consumables and/or accessories, time-consuming long and multiple processes, labor cost and use of expensive safe disposal as pollutants.

Several methods of extraction and purification of DNA or RNA and other forms of nucleic acids are known in the art to overcome these challenges. Some of these include alcohol precipitation, salting out method, organic solvent extraction, Caesium Chloride density gradients, Chelex 100 resin adsorption, silica-based binding technologies, magnetic beads-based separation, anion exchange column technology, gel exclusion chromatography, etc.

### Objectives/Need for the Invention

The above challenges warrant an ideal extraction method or kit for nucleic acid extraction. An ideal Nucleic Acid Extraction Method should enable efficient extraction of Nucleic Acids from the sample in sufficient amounts for use in downstream processes, by ensuring the minimum contaminants yielding Nucleic Acids of high quality and high purity in a consistent manner. The method also should be user-friendly with fewer steps and also less expensive cutting labor costs and processing time because Nucleic Acid Extraction is the bottleneckin the field of Molecular Biology, especially for subsequent analysis such as PCR, Sequencing, and Cloning technologies for improving the quality of human life and the beings in the planet. However, as described earlier in [0003], the mentioned challenges, and several limitations, but not limited to those posed in the current purification methods and desired features warrant improvement in the current purification methods.

The present disclosure describes an improved method for the extraction of biomolecules, including nucleic acids, from various samples. This method addresses most of the challenges outlined in the background information.

This includes the extraction of nucleic acids with desirable yield and quality suitable for direct amplification. The disclosed method employs specific buffer compositions for nucleic acid extraction and includes a kit for efficiently extracting all types of DNA, RNA, or a combination thereof from a wide range of test samples.

This invention addresses the above challenges by bringing novelty and improvement in the methods, kits, and reaction mixtures for extraction and amplifying nucleic acids of all classes of nucleic acids both naturally occurring from all types of biological samples or their modifications and chemically synthesized forms. Samples include but are not limited to plants, animals, and microorganisms.

The objective of the invention is to provide a method for the Extraction and Separation of Nucleic Acids from all biological sources comprising plants, animals, microorganisms, and fungi including the organelle DNA such as mitochondrial DNA from human/animal samples like blood and chloroplast DNA or plastid DNA from plants or food. In various embodiments of the process of extraction and separation of nucleic acids, the biological sample comprises plants, animals, fungi, and microorganisms including gram-positive bacteria, gram-negative bacteria, viruses, etc., as well as cultures obtained from soil, water, sewage, etc. The biological samples include body fluids such as blood, urine, saliva, tears, sweat, cerebrospinal fluid, etc., stool samples as well as tissue swabs.

Treatment with pre-wash buffer compositions increases the quantity and quality of the nucleic acids extracted by the process. The pre-wash buffer composition serves as a microbial enrichment medium, sample collection, storage and preservation medium, nucleic acid extraction enhancer medium, buffer to remove inhibitors, and also as a microbial lysis enhancer solution.

In various embodiments of the process of extraction and separation of nucleic acids, the process further comprises the addition of isolated nucleic acids either directly or after subjecting to dilution by 10 or 100 times with elution buffer as required in the application.

In various embodiments, depending on the source of the biological sample, the pre-wash buffer composition functions as a microbial enrichment medium, sample collection, storage and preservation medium, nucleic acid extraction enhancer medium, buffer to remove inhibitors, and also as a microbial lysis enhancer solution.

The nucleic acids extracted by the claimed process do not hamper amplification by PCR as evidenced by lesser CT (Cycle Threshold) mean value for the nucleic acids extracted using the process of the present invention when compared with other commercial kits useful for nucleic acid extraction (CT mean values) are inversely related to the amount of target nucleic acid that is present in a sample and correlates to the number of target copies present in the sample. Lower CT mean values (typically below 29 cycles) indicate high amounts of the target nucleic acid. Higher CT mean values (above 38 cycles) indicate lower amounts of target nucleic acid.

Furthermore, modifications or different embodiments of the method can be tailored based on the different sample types and the different applications in the field of Molecular Biology.

### DETAILS OF THE INVENTION

An aspect of the invention pertains to the collection and storage of biological samples, and separation of nucleic acids from these biological samples using one or more of a chemical solution(s), the composition of the chemical solutions, the process of using the solutions in a unique order and all the components as a kit, as given here.

The invention described here is not limited only to the particular sample type or a unique challenge but can be used with flexibility using different combinations of the embodiments. A few examples are shown for making various changes with equivalents or with substitutions without deviating from the scope of the invention.

The present application does not claim priority from any other patent application(s).

In some embodiments, the novel which is a cloudy precipitated buffer formulation which does not become even when heated still allows One-Step minimal sample purification for direct amplification of the nucleic acids (FIG. 1). This CP Solution solution is used in a condition such that even when heated will remain cloudy and not be clear unless organic solvents are added. The CP Solution can be used even in the cloudy precipitated state. Wherever required, further purification can also be continued from the crude extract. The invention gives a high yield of nucleic acids and quality in one single step or comprises a specific sequence of multiple steps involving the above-said samples with various buffer compositions at optimized conditions. The invention also discloses various buffer compositions which are employed in the process for the extraction of nucleic acids and a kit comprising of the above-said buffer compositions for the extraction of nucleic acids. The invention is that if done direct or purified shows either the same level of quality or even better. This is due to the fact that the loss of nucleic acids is very minimal in this method and results in high molecular weight fragments.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, numbers indicate like parts throughout the views. Additionally, a reference to the singular includes a reference to the plural unless otherwise stated or inconsistent with the disclosure here in. Throughout the specification and claims, the terms like CP Solution or OM Buffer or Solutions take the meanings of the lysis buffer with one or the different embodiments with different compositions.

An aspect of the invention pertains to a process for the extraction and separation of nucleic acids from a biological sample comprising:
The Cloudy-Precipitated Solution serves as a single extraction buffer (lysis as well), where the sample is mixed with the CP solution and centrifuged to separate the nucleic acids from the impurities. The separated nucleic acids can be used for any further application.

However, the CP solution and the method (One-to Multiple (OM)) are not limited to extraction alone, but can also be used as a collection medium, storage medium, lysis buffer, and extraction buffer based on the application of all molecules.

Another aspect of the invention pertains to buffer compositions which are employed in the process of extraction and separation of nucleic acids. The CP and One-to Multiple (OM) Process comprises a unique combination of salts, chaotropic agents, detergents, surfactants, precipitating agents, solvents, etc. The CP Solution is therefore not clear and remains cloudy with a precipitated appearance with and without samples, or even when heated to any higher temperature unless organic solvents are added to clear it. However, unlike other lysis buffers, this CP Solution lyse the cells and extracts nucleic acids and other biomolecules, even in the cloudy precipitated state. In the prior art, “in certain embodiments, kits further comprise a second surfactant, a salt, organic extraction agent(s), organic precipitating agent” (U.S. Pat. No. 6,762,027) are used in nuclear extraction. Here, the second addition is avoided. Moreover, “SDS is easily precipitable in the presence of potassium salts and generally is not added to guanidinium buffers, as it has very low solubility in high-salt, chaotropic solutions” (Robert E. Farrell).

There are several embodiments of CP Solution in One-to Multiple Process composition comprising one or more anionic, cationic and non-ionic detergents wherein the anionic surfactant is selected from the group consisting of Cholic Acid, Sodium Dodecyl Sulphate (SDS), salts thereof and combinations thereof and the non-ionic surfactant is selected from the group consisting of Polysorbates, Triton, Tweens, Polyoxyethylene Ethers, Phenyl Ethylene Glycols as preservatives, Ethylene Oxides, Castor Oil and its derivatives like but not limited to Castor Oil Ethoxylate, Poly-Hydrogenated Castor Oil, Sulfonated Castor Oil, Ethoxylated Alkyl Phenol (NP-40), Lauryl Alcohol Ethoxylate, Polyoxyethylene (20) Cetyl Ether, N,N-Bis [3-(D-Gluconamido) Propyl]-Cholamide (bigCHAP)) Sorbitan Monooleate, Sodium Deoxycolate, salts like but not limited to Sodium Acetate or Potassium Acetate, Ammonium Acetate, Lithium Acetate, Sodium Acetate, Sodium Chloride, Potassium Chloride, Lithium Acetate, Potassium Phosphate, Potassium Acetate, Sodium Chloride, Sodium Citrate, Sodium Salicylates, Diammonium Hydrogen Phosphate, Sodium Phosphate, and Sodium Sulphate, Potassium Phosphate, Sodium Citrate, Sodium Salicylates, Diammonium Hydrogen Phosphate, Sodium Phosphate, and Sodium Sulphate, charcoal, chaotropic agents like but limited to Potassium Iodide, Sodium Thiocyanate, Guanidine Thiocyanate, Urea, Guanidine Carbonate, Guanidine Phosphate, Guanidine Hydrochloride, Sodium Iodide, Thiourea, oils, organic solvents, adsorbents, Tween 20, Triton, Alcohols, Polyvinyls, adsorbents and absorbents, powdered charcoal, agar and its derivatives, agarose and pectin in powdered form, gelatin, collagen, PVP, Sodium Azide, Polysorbates, Polyoxyethylene Ethers, Phenyl Ethylene Glycols, Polyvinyls, Castor Oil Hydrogenated, Sulphonoted or Esterified, Polyvinyl and its derivatives and substitutes like but not limited to Polyvinyl Chloride, Polyvinyl Acetate, Polyvinylpyrrolidone, Polyvinylpolypyrrolidone, Acrylate, Diethanolamine derivatives like but not limited to DEA-Cetyl Phosphate, DEA Oleth-3 Phosphate, Cocamide Diethanolamine, Lauramide DEA etc. TRIS, MOPS, MES, CHAPS. Toluene derivatives like but are not limited to Benzyl- and Dibenzyl-Toluene, Butylated Hydroxy Toluene.polystyrene, polyacrylamide, nylon, silica, a woodchip, activated charcoal, an aluminum oxide, a diatomaceous earth (e.g., Celite), a cellulose material, a controlled pore glass, a siliconized glass bead, or a combination thereof.

Precipitation Solution composition comprising of salts, Guanidinium Isothiocyanate or Guanidinium Hydrochloride, 1-5M of Sodium Acetate or Potassium Acetate, Ammonium Acetate, Lithium Acetate, Sodium Acetate, Sodium Chloride, Potassium Chloride, Tween, Triton, Alcohols.

Binding Buffer composition comprising anionic, cationic, and non-ionic detergents, salts, chaotropic agents, salts, Guanidinium Isothiocyanate or Guanidinium Hydrochloride, 1-5M of Sodium Acetate or Potassium Acetate, Ammonium Acetate, Lithium Acetate, Sodium Acetate, Sodium Chloride, Potassium Chloride, Tween, Triton, Alcohols.

Wash Buffer 1 comprising anionic, cationic, and non-ionic detergents, Guanidinium Isothiocyanate or Guanidinium Hydrochloride, 1-5M of Sodium Acetate or Potassium Acetate, Ammonium Acetate, Lithium Acetate, Sodium Acetate, Sodium Chloride, Potassium Chloride, Tween, Triton, Salts, Chaotropic Agents, Salts, Alcohols.

Wash Buffer 2 comprising a C1-C10 Alcohol. 70% Ethanol, 1 to 250 mM Citrate Buffer, and 10 to 300 mM Phosphate Buffer.

Elution Buffer comprising Tris, EDTA, EGTA, 1 to 25 mM Tris, 1 to 20 mM EDTA, 1 to 50 mM EGTA.

Prewash Buffer comprising a C1-C10 Alcohol or its combination is used. 30% Methanol, 10% Ethanol, 60% Butanol, and 10 to 300 mM Phosphate Buffer.

Composition of a model CP Solution but not limited to 0.1× to 10× concentrated buffer of the below can be used as follows: 1 to 25 mM Tris, 1 to 30 mM EDTA, 1 to 50 mM EGTA, 0.01% to 5% CTAB, 0.1 to 6% SDS, 10 to 1000 mM Sodium Salicylate, 10 to 2500 mM Potassium Acetate, 10 to 4000 mM Ammonium Acetate, 10 to 4000 mM Lithium Acetate, 1 to 1000 mM Sodium Citrate, 10 to 4000 mM Sodium Chloride, 10 to 5000 mM Guanidine HCL, 0.01% to 5% Charcoal, 0.01% to 5% Tween-20, 0.01% to 6% Triton-x100, 0.01% to 5% PVP, 0.01% to 2% PVPP, 0.01% to 1% Sodium Azide, 1 to 1000 mM Sodium Hydrogen Phosphate, 1 to 500 mM Di Sodium Hydrogen Phosphate and 1 to 100 mM Ammonium Sulphate, 1 to 100 mM Potassium Phosphate, 0.01 to 25% Chloroform, 0.01 to 25% Hexane, 0.01 to 25% Phenol.

Another embodiment of one of the several alternative compositions of CP Solution but not limited to 0.1× to 10× concentrated buffer of the below can be used as follows: 0.01% to 3% Lauramide DEA, 0.01% to 1% Butylated Hydroxytoluene, 1 to 25 mM Tris, 1 to 30 mM EDTA, 0.1 to 6% SDS, 10 to 2500 mM Potassium Acetate, 10 to 4000 mM Ammonium Acetate, 10 to 4000 mM Sodium Chloride, 0.01% to 2% Sodium Deoxycholate, 0.01% to 5% Tween-20, 0.01% to 6% Triton-x100, 10 to 5000 mM Guanidine HCL, 0.01% to 5% SulfonatedCastor Oil, 0.01 to 25% Chloroform, 0.1% to 10% agar or its derivatives, Polyvinylpyrrolidone, Polyvinylpolypyrrolidone, 0.01% to 5% PVP, 0.01% to 2% PVPP, 0.01% to 1% Sodium Azide.

CP Solution and along with Anionic Surfactants: Lithium dodecyl sulfate, Sodium octyl sulfate., Sodium pentanesulfonate, Sodium dodecyl sulfate, Sodium decyl sulfate, Sodium dodecylbenzenesulfonate, Sodium stearate, Magnesium stearate, Sodium allylsulfonate, Sulfonated castor oil, Sodium ethyl 2-sulfolaureate, Sodium diisobutyl sulfosuccinate, Dodecylbenzenesulfonic acid sodium salt, Sodium lignosulfonate, Sodium lauryl polyoxyethylene ether sulfate, Sodium nonylphenol polyoxyethylene ether sulfate, Sodium lauryl sulfate, Sodium oleyl sarcosinate, Sodium pyrrolidone carbonate, Sodium polyalkyl phenyl polyoxyethylene ether sulfate, Sec-alkyl sodium sulfate, Linear alkylbenzene sulfonates, Sodium n-octylsulfonate, Sodium poly [(naphthaleneformaldehyde) sulfonate], Sodium diamyl sulfosuccinate.

Cationic Surfactants: Decyltrimethylammonium chloride, Cetyltrimethylethylammonium bromide, Dodecyl phenyl ammonium sulfate, Ammonium lauryl sulfate, Ammonium dodecylbenzenesulphonate.

Nonionic Surfactants: Butylnaphthalenesulfonic acid sodium salt, Lignosulfonic acid, calcium salt, 1-Dodecanesulfonic acid sodium salt, 3-(N,N-Dimethylpalmitylammonio) propanesulfonate, Disodium methylenebisnaphthalenesulfonate, Cleaner for heat-transfer oil furnace, LY 171883, Silk softener, Hydroxyaluminum distearate, Polyethylene, Propyleneglycol (beta-Naphthyl) (3-Sulfopropyl) Diether, Potassium Salt, 2-Dodecylbenzenesulfonate, Dicyclohexyl sulfosuccinate sodium salt, Disodium 4-dodecyl-2,4′-oxydibenzenesulfonate, Organosilicon surfactant, Sulfonated aliphatic polyester, Sodium-N-methyl-N-oleyl taurate, Dihexyl sodium sulfosuccinate, Dibasic lead stearate, Sodium methyl cocoyl taurate, Dodecyl triethanolamine sulfate, Manganous stearate, Calcium dodecylbenzene sulfonate, Disodium 4-[2-[(1-oxoundec-10-enyl) amino]ethyl] 2-sulfonatosuccinate, Fluorocarbon surfactant, Lamepon A, 1-Hexadecanesulfonic acid sodium salt, Surfactant, 1-Pentanesulfonic acid sodium salt monohydrate, Cocamidopropyl betaine, Amidoaminosurfactants, Jiuma plate amino-acid surfactant, Cleaner LS, 2,6-Dimorpholin-4-ylpyrimidine-4-carboxylic acid, C\hat{ }{12\hat{ }} fatty alcohol polyoxyethylene ether ammonium sulfate, Stearyltoluene sodium sulfonate, Nonylphenyl polyoxyethylene ether sulfate triethanolamine, Sopa, Dispersing agent CNF, Sulfate AEC, Water-decreasing agent AF, Antistatic finish agent for synthetic fiber, Frothing agent K14, Glyceryl ethercarboxylic acid salt, Calcium stearyl lactate, Monoethanolamine dodecyl sulfate, Alkoxy ethanolamido sulfosuccinate sodium salt.

An embodiment of several alternative compositions of Precipitation Buffer but not limited to 0.1× to 10× concentrated buffer of the below can be used as follows: 1-2 M of Sodium Acetate, 1-5M Potassium Acetate, 1-3 M Ammonium Acetate, 1-4 M Lithium Acetate, 1-5 M Sodium Chloride, 1-2 M Potassium Chloride.

An embodiment of several alternative compositions of Binding Buffer but not limited to 0.1× to 10× concentrated buffer of the below can be used as follows: 1M to 9 M Guanidinium Isothiocyanate and or 1M to 9 M Guanidinium Hydrochloride, 0.1-1 M of Sodium Citrate, 0.1-1 M of Phosphate Buffer, 1% to 20% Polyoxyethylene Ethers, Sodium Acetate, Sodium Chloride, Potassium Chloride, 1% to 50% tween 20, 1% to 20% Triton, 1% to 30% Sulfonated Castor Oil.

An embodiment of several alternative compositions of Wash Buffer but not limited to 0.1× to 10× concentrated buffer of the below can be used as follows: 1M to 9 M Guanidinium Isothiocyanate and or 1M to 9 M Guanidinium Hydrochloride, 0.1-1 M of Sodium Citrate, 0.1-1 M of Phosphate Buffer, Sodium Acetate, Sodium Chloride, Potassium Chloride, 1% to 50% Tween 20, 1% to 20% Triton, 1% to 30% Sulfonated Castor Oil.

An embodiment of several alternative compositions of Pre-Wash Buffer but not limited to 0.1× to 10× concentrated buffer of the below can be used as follows: 1% to 70% Xylene, 10 to 70% Butanol, 10 to 70% Methanol, 10 to 70% Isopropanol, 10 to 300 mM Phosphate Buffer.

Use of polymerases that is resistant to inhibitors along with CP solution and OM method.

FIG. 1: Flowchart showing extraction of biomolecules like nucleic acids using CP Solution in OM Method and kits.

Cloudy Precipitated (CP) Solution for Extraction of All Biomolecules Including Nucleic Acids.

The Cloudy Precipitated (CP) solution remains cloudy and does not clear, even when heated with or without a sample.

Unlike other lysis buffers, the CP Solution lyse the cells and extracts nucleic acids and other biomolecules, even in the cloudy precipitated state.

FIG. 1 Part 1—Empty Tube: Prepare an empty tube.

FIG. 1 Part 2—CP Solution: Add the CP solution to the tube.

FIG. 1 Part 3—Add Sample: Introduce the sample into the CP solution.

FIG. 1 Part 4—Heat: Heat the mixture.

FIG. 1 Part 5—Centrifuge: Centrifuge the heated mixture.

FIG. 1 Part 6—Purified Biomolecules: Collect the purified biomolecules from the supernatant.

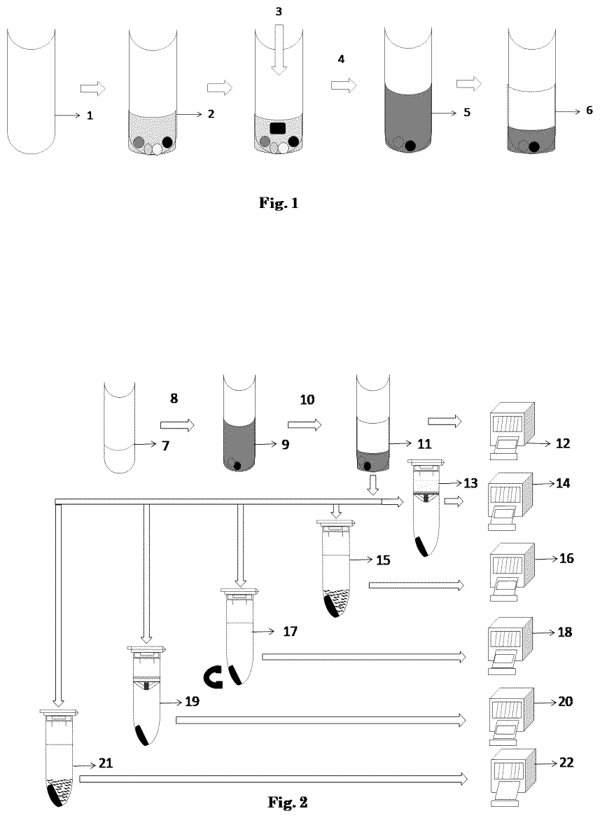

FIG. 2: Flowchart showing choices of all different Embodiments used in the process of extraction of nucleic acids.

Based on the level of purity of nucleic acid in the one minute method, the single CP Solution can be used alone in one step or can be further subjected to additional chemicals and steps for the desired purity.

FIG. 2 Part 7—Sample: Collect the sample.

FIG. 2 Part 8—Pre-treatment: Pre-treat the sample to prepare it for the extraction of nucleic acids.

FIG. 2 Part 9—CP Solution: Add the CP solution.

FIG. 2 Part 10—Tissue Homogenization, Heat, and Freeze: Homogenize the mixture, apply heat, and freeze.

FIG. 2 Part 11—Centrifuge: Centrifuge to obtain the purified nucleic acids from the supernatant.

FIG. 2 Part 12—Biological Applications: Utilize the extracted nucleic acids from the supernatant for various biological applications.

FIG. 2 Part 13—Filter Membranes & Sephadex: Use filters membranes and Sephadex for further purification of nucleic acids from the supernatant.

FIG. 2 Part 14—Biological Applications: Utilize the extracted nucleic acids for various biological applications.

FIG. 2 Part 15—Precipitation: Use precipitation for further purification of nucleic acids from the supernatant.

FIG. 2 Part 16—Biological Applications: Utilize the extracted nucleic acids for various biological applications.

FIG. 2 Part 17—Magnetic silica beads: Use Magnetic silica beads for further purification of nucleic acids from the supernatant.

FIG. 2 Part 18—Biological Applications: Utilize the extracted nucleic acids for various biological applications.

FIG. 2 Part 19—Silica matrix: Use spin column for further purification of nucleic acids from the supernatant.

FIG. 2 Part 20—Biological Applications: Utilize the extracted nucleic acids for various biological applications.

FIG. 2 Part 21—Silica powder (silica gel): Use silica powder (silica gel) for further purification of nucleic acids from the supernatant.

FIG. 2 Part 22—Biological Applications: Utilize the extracted nucleic acids for various biological applications.

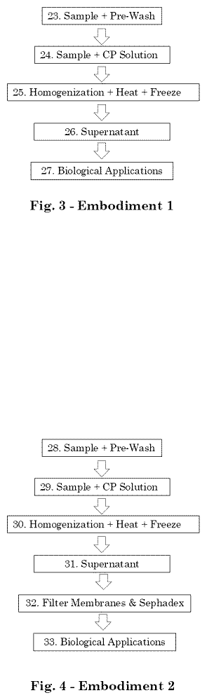

FIG. 3—Steps—Embodiment 1

FIG. 3 Part 23—Add the pre-treatment solution to the sample and vortex well. Centrifuge the mixture, then perform one or two pre-wash steps. Proceed with the following steps.

FIG. 3 Part 24—Add the pre-washed sample to the CP Solution and vortex to mix well.

FIG. 3 Part 25—Perform bead-beating or any suitable method for homogenization. Optionally, treat with proteinase K, RNAse, or DNAse, and heat inactivate for 10 minutes at 25 to 95° C., depending on the sample type, enzyme nature, and nucleic acid criteria. Incubate the mixture at 10° C. to 95° C. for a few minutes to a few hours. Keep the mixture at −20° C. or on ice for a few minutes to a few hours.

FIG. 3 Part 26—Centrifuge to remove impurities.

FIG. 3 Part 27—Use the crude supernatant for biological applications. Store the supernatant containing nucleic acids at −20° C. or −80° C.

FIG. 4—Steps—Embodiment 2

FIG. 4 Part 28—Add the pre-treatment solution to the sample and vortex well. Centrifuge the mixture, then perform one or two pre-wash steps. Proceed with the following steps.

FIG. 4 Part 29—Add the pre-washed sample to the CP Solution and vortex to mix well.

FIG. 4 Part 30—Perform bead-beating or any suitable method for homogenization. Optionally, treat with proteinase K, RNAse, or DNAse, and heat inactivate for 10 minutes at 25 to 95° C., depending on the sample type, enzyme nature, and nucleic acid criteria. Incubate the mixture at 10° C. to 95° C. for a few minutes to a few hours. Keep the mixture at −20° C. or on ice for a few minutes to a few hours.

FIG. 4 Part 31—Centrifuge to remove impurities.

FIG. 4 Part 32—Pass the crude supernatant through filter membranes, Sephadex, a gel chromatography column with beads or resin, or any other absorbent material, such as charcoal, to remove salts and impurities. Collect the flow-through.

FIG. 4 Part 33—The flow-through containing the nucleic acids can be used for biological applications. Store the supernatant containing nucleic acids at −20° C. or −80° C.

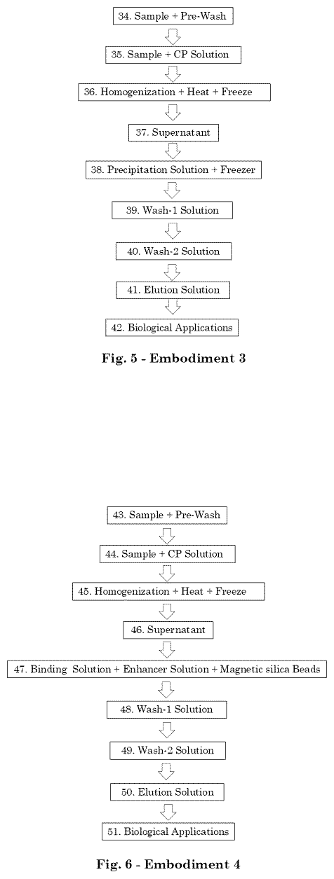

FIG. 5—Steps—Embodiment 3

FIG. 5 Part 34—Add the pre-treatment solution to the sample and vortex well. Centrifuge the mixture, then perform one or two pre-wash steps. Proceed with the following steps.

FIG. 5 Part 35—Add the pre-washed sample to the CP Solution and vortex to mix well.

FIG. 5 Part 36—Perform bead-beating or any suitable method for homogenization. Optionally, treat with proteinase K, RNAse, or DNAse, and heat inactivate for 10 minutes at 25 to 95° C., depending on the sample type, enzyme nature, and nucleic acid criteria. Incubate the mixture at 10° C. to 95° C. for a few minutes to a few hours. Keep the mixture at −20° C. or on ice for a few minutes to a few hours.

FIG. 5 Part 37—Centrifuge to remove impurities.

FIG. 5 Part 38—Add precipitation solution to the crude supernatant, mix well, freeze the mixture, and centrifuge. Discard the supernatant.

FIG. 5 Part 39—Add Wash Buffer-1 to the pellet, mix, and centrifuge. Discard the supernatant.

FIG. 5 Part 40—Add Wash Buffer-2 to the pellet, mix, and centrifuge. Discard the supernatant and air dry the pellet.

FIG. 5 Part 41—Add Elution Buffer to the pellet and mix well.

FIG. 5 Part 42—Use the purified nucleic acids for biological applications. Store the supernatant containing nucleic acids at −20° C. or −80° C.

FIG. 6—Steps—Embodiment 4

FIG. 6 Part 43—Add the pre-treatment solution to the sample and vortex well. Centrifuge the mixture, and then perform one or two pre-wash steps. Proceed with the following steps.

FIG. 6 Part 44—Add the pre-washed sample to the CP Solution and vortex to mix well.

FIG. 6 Part 45—Perform bead-beating or any suitable method for homogenization. Optionally, treat with proteinase K, RNAse, or DNAse, and heat inactivate for 10 minutes at 25 to 95° C., depending on the sample type, enzyme nature, and nucleic acid criteria. Incubate the mixture at 10° C. to 95° C. for a few minutes to a few hours. Keep the mixture at −20° C. or on ice for a few minutes to a few hours.

FIG. 6 Part 46—Centrifuge to remove impurities.

FIG. 6 Part 47—Add binding solution and enhancer solution to the crude supernatant, mix well. Load the supernatant onto silica magnetic beads, mix well, and separate the magnetic beads using a magnetic separator. Discard the supernatant by pipetting.

FIG. 6 Part 48—Add Wash Buffer-1 to the silica magnetic beads, mix well, and separate the magnetic beads using a magnetic separator. Discard the supernatant by pipetting.

FIG. 6 Part 49—Add Wash Buffer-2 to the silica magnetic beads, mix well, and separate the magnetic beads using a magnetic separator. Discard the supernatant by pipetting.

FIG. 6 Part 50—Add Elution Buffer to the silica magnetic beads, incubate at room temperature or at 65° C. for 5 to 10 minutes, and separate the magnetic beads using a magnetic separator. Pipette the eluted supernatant into a new tube.

FIG. 6 Part 51—Use the purified nucleic acids for biological applications. Store the supernatant containing nucleic acids at −20° C. or −80° C.

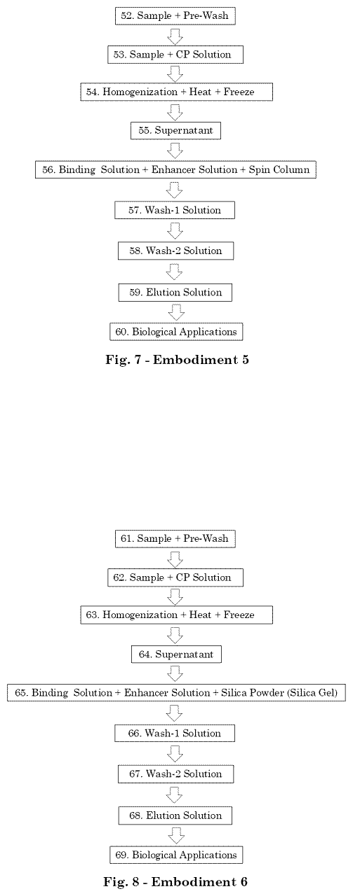

FIG. 7—Steps—Embodiment 5

FIG. 7 Part 52—Add the pre-treatment solution to the sample and vortex well. Centrifuge the mixture, and then perform one or two pre-wash steps. Proceed with the following steps.

FIG. 7 Part 53—Add the pre-washed sample to the CP Solution and vortex to mix well.

FIG. 7 Part 54—Perform bead-beating or any suitable method for homogenization. Optionally, treat with proteinase K, RNAse, or DNAse, and heat inactivate for 10 minutes at 25 to 95° C., depending on the sample type, enzyme nature, and nucleic acid criteria. Incubate the mixture at 10° C. to 95° C. for a few minutes to a few hours. Keep the mixture at −20° C. or on ice for a few minutes to a few hours.

FIG. 7 Part 55—Centrifuge to remove impurities.

FIG. 7 Part 56—Add binding solution and enhancer solution to the crude supernatant, mix well. Load the supernatant onto a silica matrix-based spin column and centrifuge. Discard the flow-through.

FIG. 7 Part 57—Add Wash Buffer-1 to the silica matrix-based spin column and centrifuge. Discard the flow-through.

FIG. 7 Part 58—Add Wash Buffer-2 to the silica matrix-based spin column and centrifuge. Discard the flow-through. Air dry the silica matrix-based spin column.

FIG. 7 Part 59—Add Elution Buffer to the silica matrix-based spin column, incubate at room temperature or at 65° C. for 5 to 10 minutes, and centrifuge. Save the flow-through.

FIG. 7 Part 60—Use the purified nucleic acids for biological applications. Store the supernatant containing nucleic acids at −20° C. or −80° C.

FIG. 8—Steps—Embodiment 6

FIG. 8 Part 61—Add the pre-treatment solution to the sample and vortex well. Centrifuge the mixture, then perform one or two pre-wash steps. Proceed with the following steps.

FIG. 8 Part 62—Add the pre-washed sample to the CP Solution and vortex to mix well.

FIG. 8 Part 63—Perform bead-beating or any suitable method for homogenization. Optionally, treat with proteinase K, RNAse, or DNAse, and heat inactivate for 10 minutes at 25 to 95° C., depending on the sample type, enzyme nature, and nucleic acid criteria. Incubate the mixture at 10° C. to 95° C. for a few minutes to a few hours. Keep the mixture at −20° C. or on ice for a few minutes to a few hours.

FIG. 8 Part 64—Centrifuge to remove impurities.

FIG. 8 Part 65—Add binding solution and enhancer solution to the crude supernatant, mix well. Load the supernatant onto silica powder (silica gel) and centrifuge. Discard the flow-through.

FIG. 8 Part 66—Add Wash Buffer-1 to the silica powder (silica gel) and centrifuge. Discard the flow-through.

FIG. 8 Part 67—Add Wash Buffer-2 to the silica powder (silica gel) and centrifuge. Discard the flow-through. Dry heat the silica powder (silica gel) at 65° C. for 5 minutes.

FIG. 8 Part 68—Add Elution Buffer to the silica powder (silica gel), incubate at room temperature or at 65° C. for 5 to 10 minutes, and centrifuge. Save the flow-through.

FIG. 8 Part 69—Use the purified nucleic acids for biological applications. Store the supernatant containing nucleic acids at −20° C. or −80° C.

TABLE 1
Extraction of Nucleic Acids from Blood Samples showing the
performance of this invention in comparison to existing methods.
DNA from Blood/Serum/Plasma Samples

| | | | | | Copy Fold |
|---|---|---|---|---|---|
| | | | Fold Better | | Better |
| | | | than the | | than the |
| | CT Value | | commercial | Copy | commercial |
| Method | PCR | Yield OD | Kit | Number | Kit2 |
| Embodiment-1 | 22.40 | 1.72 | 18 | 3691000 | 12 |
| Commercial Kits | 25.88 | 0.09 | 1 | 318900 | 1 |
| Embodiment-2 | 17.14 | 1.72 | 18 | 193900000 | 12 |
| Commercial Kits | 20.25 | 0.09 | 1 | 16840000 | 1 |
| Embodiment-3 | 19.80 | 2.96 | N/A | 10040000 | N/A |
| Commercial Kits | Inhibited | Inhibited | Inhibited | Inhibited | Inhibited |
| Embodiment-5 | 21.18 | 0.06 | 0.65 | 8522000 | 3 |
| Commercial Kits | 22.56 | 0.09 | 1.00 | 3189000 | 1 |

Embodiment-1 and Embodiment-2 significantly outperformed the commercial kits in terms of Yield OD, CT Value, and Copy Number, showing up to 18-fold and 12-fold improvement, respectively.

Embodiment-3 also showed a high yield and copy number, but the commercial kits were inhibited, making direct comparison challenging.

Embodiment-5 showed slight improvement over the commercial kits in terms of copy number, though its yield was lower.

TABLE 2
PCR of Extracted Nucleic Acids from Stool Samples showing the
performance of this invention in comparison to existing methods.
Table-6a 16ss PCR of DNA from Stool Samples

| | | | Yield OD | | Copy Number |
|---|---|---|---|---|---|
| | | | Fold Better | | Fold Better |
| | CT | | than the | | than the |
| | Value | Yield | commercial | Copy | commercial |
| Method | PCR | OD | Kit | Number | Kit |
| Embodiment-1 | 14 | 4 | So no | 10190000 | So no |
| | | | comparison | | comparison |
| Commercial Kits | N/A | N/A | N/A | N/A | Inhibited |
| Embodiment-2 | 24 | N/A | So no | 42120 | So no |
| | | | comparison | | comparison |
| Commercial Kits | N/A | N/A | N/A | N/A | Inhibited |
| Embodiment-3 | 13 | 14 | So no | 24590000 | 59 |
| | | | comparison | | |
| Commercial Kits | 20 | 0 | N/A | 409500 | 1 |
| Embodiment-5 | 16 | 1 | So no | 2546000 | 7 |
| | | | comparison | | |
| Commercial Kits | 20 | 0 | N/A | 310100 | 1 |

Embodiment-1 and Embodiment-3 showed significantly lower CT values and higher yields, with Embodiment-3 achieving a notable 59-fold increase in copy number compared to the commercial kits.

Embodiment-2 and Embodiment-5 also showed improved performance, though the commercial kits were inhibited, making direct yield comparisons difficult.

Commercial kits consistently failed to produce results, indicated by inhibited values, making the embodiments significantly more effective for DNA extraction from stool samples.

1. A One-to-Multiple Method (OM) for isolating nucleic acid from any biological sample in one or multiple processes, using a CP Buffer solution that has a composition that is not clear and remains cloudy giving a precipitated appearance even when heated or not heated to high temperatures with or without the addition of sample, unless organic solvents are added. Unlike other buffer solutions, this buffer extractions even in cloudy precipitated conditions.

2. The CP Buffer of claim 1, further comprising of one or more of the adsorbents and absorbents, powdered charcoal, agar and its derivatives, agarose, and pectin in powdered form along with salts, chaotropic agents, and detergents, organic solvents in unclear, cloudy, and precipitated form.

3. The method of claims 1 and 2, comprises: (a) mixing the biological sample containing nucleic acids with the prefilled single CP Buffer in tubes or adding a single CP buffer to the sample in a tube to obtain a Mixture-A; allowing to settle the impurities over time or centrifuging the Mixture-A to obtain a supernatant (Mixture-B) comprising the nucleic acids and or other biomolecules.

4. The CP Buffer of claims 1 and 2 comprises anionic detergents at 0.1 to 6% concentration along with 10 to 2000 mM concentration of any one or more of neutralizing or precipitate-forming salts, adsorbents, absorbents, and chaotropic agents.

5. The CP Buffer of claims 1 and 2 comprises cationic detergents at 0.1 to 6% concentration along with 10 to 2000 mM concentration of any one or more of neutralizing or precipitate-forming salts, adsorbents, absorbents, and chaotropic agents.

6. The CP Buffer of claims 1 and 2 comprises cationic and anionic detergents at 0.1 to 6% concentration along with 10 to 2000 mM concentration of any one or more neutralizing or precipitate-forming salts, adsorbents, absorbents, and chaotropic agents.

7. The CP Buffer of claims 1 and 2 comprises chaotropic agents at 0.1 to 8 M concentration along with 10 to 2000 mM concentration of any one or more of neutralizing or precipitate-forming salts, and anionic detergents.

8. The method of claims 1, 2,3,4,5,6, and 7 further comprised wherein said nucleic acids are isolated in Multiple Steps or with additional steps, the additional steps comprising i) by passing the Mixture-A or Mixture-Bonto an adsorbent matrix column but not limited to charcoal, chelax, resins, PVP, PVPP, oils, Sephadex, and collecting the resulting nucleic acid solution.

9. The method of claims 1,2,3,4,5,6 and 7 further comprised wherein said nucleic acids are isolated in multiple steps or with additional steps, the additional steps comprising i) by passing the Mixture-A or Mixture-Bon to a gel chromatography column but limited to Sephadex and collecting the resulting nucleic acid solution.

10. Subject to the resultant product in claims 3 and 4 to freeze drying or alcohol precipitation with salt for concentrating the nucleic acids.

11. An extraction kit employing the buffers and process flow in claims 1, 2,3,4,5,6,7,8,9,10 further includes instructions for isolating RNA or total RNA, micro-RNA, cell-free RNA, and fragmented RNA, from a biological sample.

12. The kit of claim 11 further includes instructions for isolating DNA or total DNA, cell-free DNA, and fragmented DNA from a biological sample.

13. The kit of claim 11 further includes instructions for isolating plasmid DNA from a biological sample.

14. The method of claims 1,2,3,4,5,6,7 and 11 is further comprised wherein said nucleic acids are isolated from an extraction solution in a single step.

15. claims 1,2,3,4,5,6,7 and 11 wherein admixing the sample to form a homogenate where all these reactions occur in One Step and help in the separation of nucleic acids.

16. The method of claims 1,2,3,4,5,6,7 and 11 wherein said biological specimen is a Gram-positive bacterium.

17. The method of claims 1,2,3,4,5,6,7 and 11 wherein said biological specimen has polyphenols.

18. The method of claims 1,2,3,4,5,6,7 and 11 wherein the biological specimen is a clinical isolate of a microorganism, wherein the microorganism is a bacterium, a virus, a fungus, or a combination thereof.

19. The method of claims 1,2,3,4,5,6,7 and 11 wherein the biological specimen is obtained from a human, animal, plant, or microbe.

20. The method of claims 1,2,3,4,5,6,7 and 11 wherein the extract also can effectively lower the TM or melting temperature in direct PCR reactions.

21. The method of claims 1,2,3,4,5,6,7 and 11 wherein the extract can be used directly for molecular applications.

22. The kit of claim 11 further includes instructions for isolating nucleic acids, proteins, fats, and all biomolecules from food or any biological sample.

23. The method of claims 1,2,3,4,5,6,7 and 11 further comprised wherein said nucleic acids are isolated in multiple steps or with additional steps, the additional steps comprising i) by adding a binding solution to the Mixture-B and allowing adsorbing said nucleic acids in Mixture-A onto silica-coated magnetic particles ii) washing said nucleic acid adsorbed silica-coated magnetic particles, with wash buffers iii) eluting said nucleic acids from said silica-coated magnetic particles into the elution solution.

24. The method of claims 1,2,3,4,5,6,7 and 11 further comprised wherein said nucleic acids are isolated in Multiple Steps or with additional steps, the additional steps comprising i) by adding a binding solution to the Mixture-B and allowing adsorbing of said nucleic acids in Mixture-B onto silica membrane. ii) Washing said nucleic acid adsorbed silica with wash buffers. iii) Eluting said nucleic acids from said silica membrane into the elution solution.

25. The method of claims 1,2,3,4,5,6,7 and 11 further comprised wherein said nucleic acids are isolated in multiple steps or with additional steps, the additional steps comprising i) by adding a precipitation solution and alcohols to the Mixture-B and allowing said nucleic acids in Mixture-B to form a precipitate by settling or on centrifugation. ii) Washing said nucleic acid with wash buffers and 75% alcohols. iii) Drying and dissolving the precipitate into the elution solution.

26. The method of claims 1,2,3,4,5,6,7 and 11 wherein the biological sample is selected from blood, sputum, serum, saliva, urine, semen, cell cultures, nasal swabs, cheek swabs, tissue extracts, and a combination thereof.

27. The method of claims 1,2,3,4,5,6,7 and 11 wherein said method includes mechanical disruption or enzymatic disintegration of cell walls of the samples, from gram-positive, gram-negative bacteria, fungi, plants, yeasts, fossils, Formalin Fixed-Paraffin-Embedded tissues (FFPE), nucleic acid from agarose or acrylamide gels, etc.

28. In various embodiments claims 1,2,3,4,5,6,7 and 11 wherein depending on the source of the biological sample, the CP Solution composition functions as a microbial enrichment medium, sample collection, transport.

29. Storage and preservation medium, nucleic acid extraction enhancer medium, buffer to remove inhibitors, and microbial enhancer solution.

30. In various embodiments claims 1,2,3,4,5,6,7 and 11 wherein depending on the source of the biological sample, the pre-wash buffer composition functions as a microbial enrichment medium, sample collection, storage and preservation medium, nucleic acid extraction enhancer medium, buffer to remove inhibitors, and microbial enhancer solution.

31. In various embodiments claims 1,2,3,4,5,6,7 and 11 wherein can be applied in a capillary flow-through system or chip-controlled system.

32. In various embodiments claims 1,2,3,4,5,6,7 and 11 wherein can be applied in a liquid handling system.

33. In various embodiments claims 1,2,3,4,5,6,7 and 11 wherein can be applied in a robotic or automation system.

34. The method of claims 1,2,3,4,5,6,7 and 11 wherein kit in an alternative embodiment claims 1,2,3,4,5,6,7 and 11 wherein it comprising of the direct buffer comprises wherein the anionic surfactant is selected from the group consisting of Caprylic Acid, Cholic Acid, Deoxycholic Acid, Glycocholic Acid, Sodium Dodecyl Sulphate (SDS), salts thereof and combinations thereof and the non-ionic surfactant is selected from the group consisting of Polysorbates, Polyoxyethylene Ethers, Phenyl Ethylene Glycols, Polyvinyls, and combinations thereof.

35. The method of claims 1,2,3,4,5,6,7 and 11 wherein CP Buffer with agar, agarose, pectin, charcoal, and combinations thereof.

36. The method of claims 1, 2,3,4,5,6,7 and 11 wherein the non-ionic surfactant is present in the reaction at a concentration in the range of about 0.001% to about 0.1% (w/v).

37. The method of claims 1,2,3,4,5,6,7 and 11 wherein the anionic surfactant is present in the reaction at a concentration that is less than or equal to about 0.1% (w/v) to 80% and the non-ionic surfactant is present in the reaction at a concentration that is less than about 50% (w/v).

38. The method of claims 1,2,3,4,5,6,7 and 11, Salts and other compositions in a concentration of from about 1 mM to about 5 M.

39. The method of claims 1,2,3,4,5,6,7 and 11, wherein said chaotropic is selected from at least one of the groups consisting of urea, and Guanidine Thiocyanate (GITC).

40. The method of claims 1,2,3,4,5,6,7 and 11 wherein said oil is non-hydrogenated, sulphonated, or esterified.

41. The method of claims 1,2,3,4,5,6,7 and 11 further comprises of alcohol wherein said alcohol is selected from at least one of the groups consisting of C1 to C12.

42. The kit of claims 1,2,3,4,5,6,7 and 11, wherein: the binding solution also comprises the following components: 1-8 M of Guanidinium Isothiocyanate or Guanidinium Hydrochloride, 1-5M of Sodium Acetate or Potassium Acetate, Ammonium Acetate, Lithium Acetate, Sodium Acetate, Sodium Chloride, Potassium Chloride, Tween, Triton.

43. The method of claims 1,2,3,4,5,6,7 and 11, wherein CP Buffer 0.1× to 10× concentrated buffer and the resulting sample extract diluted to 1 to 10,000 times and used in applications.

44. The method of claims 1,2,3,4,5,6,7 and 11, wherein Adding a washing buffer solution to the magnetic bead-nucleic acid complex, silica-matrix membrane, alcohol-precipitated pellet, and washing to remove impurities.

45. The method of claims 1,2,3,4,5,6,7 and 11, wherein one or more chaotropic agents are selected from Potassium Iodide, Sodium Thiocyanate, Guanidine Thiocyanate, Urea, Guanidine Carbonate, Guanidine Phosphate, Guanidine Hydrochloride, Aminoguanidine HCl, Sodium Iodide, Thiourea, and combinations.

46. The method of claims 1,2,3,4,5,6,7 and 11, wherein the one or more salts are selected from Ammonium Acetate, Potassium Citrate, Ammonium Phosphate, Sodium Acetate, Ammonium Sulphate, Lithium Chloride, Lithium Sulphate, Magnesium Sulphate, Magnesium Chloride, Potassium-1-Chloride, Lithium Acetate, Potassium Phosphate, Potassium Acetate, Sodium Chloride, Sodium Citrate, Sodium Salicylates, Diammonium Hydrogen Phosphate, Sodium Phosphate, and Sodium Sulphate, and combinations thereof.

47. The method of claims 1,2,3,4,5,6,7 and 11, wherein the one or more non-ionic detergents are selected from Polyoxyethylene (20) Sorbitan Monolaurate (Tween-20), Polyoxyethylene Triton X-100, Castor Oil and its derivatives like but not limited to Castor Oil Ethoxylate, Poly-Hydrogenated Castor Oil, Sulfonated Castor Oil, Ethoxylated Alkyl Phenol (NP-40), Lauryl Alcohol Ethoxylate, Polyoxyethylene (20) Cetyl Ether, N,N-Bis [3-(D-Gluconamido) Propyl]-Cholamide (bigCHAP)) Sorbitan Monooleate and combinations thereof.

48. The method of claims 1,2,3,4,5,6,7 and 11, wherein buffers have polyvinyl and its derivatives and substitutes like but not limited to Polyvinyl Chloride, Polyvinyl Acetate, Polyvinyl Alcohol, Polyvinylpyrrolidone, Polyvinylpolypyrrolidone, Acrylate, Diethanolamine derivatives like but not limited to DEA-Cetyl Phosphate, CDEA, Cocamide Diethanolamine, Lauramide DEA etc. Toluene derivatives like but are not limited to Benzyl- and Dibenzyl-Toluene, Butylated Hydroxy Toluene.

49. The method of claims 1,2,3,4,5,6,7 and 11, further comprises alcohol wherein said alcohol is selected from at least one of the groups consisting of C1 to C12.

50. The kit of claims 1,2,3,4,5,6,7 and 11, wherein: the CP binding solution also comprises the following components: 1-8 M of Guanidinium Isothiocyanate or Guanidinium Hydrochloride, 1-5 M of Sodium Acetate or Potassium Acetate, Ammonium Acetate, Lithium Acetate, Sodium Acetate, Sodium Chloride, Potassium Chloride, Tween, Triton.

51. The method of claims 1,2,3,4,5,6,7 and 11, wherein the one or more and combinations thereof CP Buffer compositions.

52. The method of claims 1,2,3,4,5,6,7 and 11, wherein the Embodiment of several alternative compositions of CP Buffer but not limited to 0.1× to 10× concentrated buffer of the below can be used.

53. The method of claims 1,2,3,4,5,6,7 and 11, wherein the Embodiment of several alternative compositions of Precipitation Buffer but not limited to 0.1× to 10× concentrated buffer of the below can be used.

54. The method of claims 1,2,3,4,5,6,7 and 11, wherein the Embodiment of several alternative compositions of Binding Buffer but not limited to 0.1× to 10× concentrated buffer of the below can be used.

55. The method of claims 1,2,3,4,5,6,7 and 11, wherein the Embodiment of several alternative compositions of Pre-Wash Buffer but not limited to 0.1× to 10× concentrated buffer of the below can be used.

56. The method of claims 1,2,3,4,5,6,7 and 11, wherein the choice of exercising of different embodiments for desirable extent of purification and sample type of nucleic acids required.

57. The method of claims 1,2,3,4,5,6,7 and 11, wherein All in one Copurification of DNA, RNA, cfDNA, cfRNA.

58. The method of using inhibitor resistant polymerases with OM purified nucleic acids.
